(19) United States
(12) Patent Application Publication
(10) Pub. No.: US 2007/0162024 A1
(43) Pub. Date: Jul. 12, 2007

(54) IMPLANT FOR ANCHORING IN BONES

(75) Inventor: Jurgen Siemonsmeier, Bonn (DE)

Correspondence Address: JOYCE VON NATZMER;HVP LLP, 200 Madison Avenue, Suite 1901, New York, NY, 10016, US

(73) Assignee: TISSUEDENT GMBH & CO. KG, Bonn (DE)

(21) Appl. No.: 11/611,207

(22) Filed: Dec. 15, 2006

Related U.S. Application Data
(60) Provisional application No. 60/750,402, filed on Dec. 15, 2005.

(51) Int. Cl. A61B 17/58

(52) U.S. Cl. 606/72

(57) ABSTRACT

The invention relates to an implant 20 comprising at least one shaft area 21 for anchoring in a bony structure and further comprising at least one opening 24 at the distal end 25 of the shaft area 21 in which the shaft area 21 has a continuous bore 23 extending from the opening 24 to at least one outlet 26 at the apical end 27, so that targeted introduction of material at least into the periapical area is possible with a stable anchoring in the bone structure even after implantation.

### CROSS REFERENCE TO RELATED APPLICATIONS

This application claims the benefit of U.S. provisional application No. 60/750,402, filed Dec. 15, 2005, which is incorporated herein by reference in its entirety.

### BACKGROUND OF THE INVENTION

The invention relates to an implant comprising at least one shaft area for anchoring in a bony structure and further comprising at least one opening at the distal end of the shaft area.

Implants of the type defined above are known in various fields of surgery and/or oral surgery and usually consist of a shaft area and a head area shaped thereon or attached thereto. Whereas the shaft area with its apical end is inserted into the osseous tissue and serves to anchor the implant in the bone, a prosthesis or a dental crown replacement is attached to the head area, for example. The shaft area is often made of a porous material or a solid material that is roughened at the surface, so that the osseous tissue can grow into the pores of the implant to improve the fixation of the implant. On its distal end, the head often has an opening or fastening means into and/or onto which the prosthesis or an adaptor provided for it may be attached. For example, there may be an inside thread in the distal opening in the head area into which a corresponding screw can be screwed. In daily dental or orthopedic surgical practice, the implant is placed as an anchoring module in the bone, i.e., being screwed in by means of a self-cutting outside thread and then one must wait for enossal integration. To improve integration into the osseous tissue, a wide variety of surface structures or material structures for the shaft area have already been investigated.

In anchoring implants in bone, there is often the problem that stable integration of the implant is impossible or may be accomplished only with great effort owing to the structure or thickness of the corresponding bone section. For example, in anchoring dental implants in the upper jaw, very little bone is available below the maxillary sinus (sinus maxillaris) for attachment of the implant because the jawbone has a relatively minor thickness at the locations below the maxillary sinus. Therefore, the jawbone must often be “widened” into the maxillary sinus to achieve an adequate bone thickness to anchor an implant. However, this method, known as a “sinus lift,” is very complex and is very unpleasant for the patient. Another problem in anchoring implants in a bony structure is that after insertion of the implant, inflammatory processes may take place in the periapical and/or peri-para-implant area, but because the implant has already been integrated, these processes cannot be treated adequately and therefore may lead to repeat surgery or even removal of the implant.

A dental root implant consisting of a head arranged at the distal end of the implant and a shaft connected to it in one piece is known from DE 41 05 165 A1. The head has contact faces running obliquely with respect to the longitudinal axis of the shaft and coming in contact with the jawbone; these contact faces have an open-celled structure into which osseous tissue can grow, so that the implant is anchored in the jaw. The head also has a distal opening that is connected to the shaft via a bore. The shaft consists of an open network through which an antiseptic can be introduced into the osseous bed. However, this known implant has the disadvantage that the shaft does not make any contribution toward anchoring the implant in the bone but instead the stability of the implant depends exclusively on the contact faces of the head and is therefore relatively low. Furthermore, administration of the antiseptic specifically in certain areas of the jaw is impossible due to the open network structure.

Furthermore, U.S. Pat. No. 6,939,135 B2, which is incorporated herein by reference, discloses a dental root implant, which also consists of a head arranged on the distal end of the implant and a shaft connected to the head in one piece with it. The cylindrical shaft has an outside thread for screwing it into the jawbone and rows of outlet openings running vertically therein with the openings extending radially outward. These outlet openings communicate with an opening at the distal end of the shaft via a channel running centrally through the shaft, so that growth factors introduced through the opening can be introduced through the outlet openings into the bone structure radially surrounding the implant. However, this known implant has the disadvantage that no material can be introduced into the periapical region to avoid the complex sinus lift mentioned above, for example.

### SUMMARY OF THE INVENTION

Therefore, the object of the present invention is to create an implant of the type defined in the introduction which allows the introduction of material specifically into the periapical region even after implantation while achieving stable anchoring in the bone structure.

According to this invention, this object is achieved by an implant of the type defined in the introduction, whereby the shaft area has a continuous bore extending from the opening up to at least one outlet at the apical end. The inventive implant allows specific administration of substances and/or penetration of material into the periapical (tip) area and/or the peri-para-implant area after implantation of the implant through the outlet situated apically to thereby avoid a complex external or internal sinus lift by controlling tissue regeneration, which is now possible post implantation. The inventive implant thus allows administration of various agents, medications, cells or other substances via the shaft area directly during surgery and/or subsequently over a long period of time in one or more additional sessions as inductive and/or adjuvant and/or therapeutic measures. Targeted administration of any substances, medications and/or cells into the periapical and/or paraimplantational area is thus made possible, while the implant itself remains in its property of “being carrier or anchor of old standards,” i.e., the possibility is retained of administering a promoting treatment post implantation for treatment of the surrounding tissue, especially at the tip of the implant, by administration of medication, e.g., antibiotics or growth factors. It is therefore possible, when using the inventive implant, to treat complications even several years post implantation in the area which would otherwise have been inaccessible in the past. Another special advantage of the invention is that tissue regeneration is made possible in the post- or peri-implantation period by administration of stem cells of any type into the periapical region.

In an advantageous embodiment of the invention, the shaft area has at least one fastening element at the apical end in the bore. For example, a line, an access tube, a screw, a tensioning spindle (mandrel) or a stopper may be attached to the fastening element. The fastening element may be an inside thread and/or part of a plug or catch connection. If a line, which may be a cannula or a tube, for example, or an access tube is attached to the fastening element, then reliable and targeted introduction or administration of substances, cells or other material through the outlet into the periapical area is advantageously possible. By means of a screw or a corresponding stopper attached to the fastening element, the outlet may also be sealed, thereby preventing the penetration of foreign bodies or microorganisms into the periapical surgical region between the individual treatments.

If the shaft area has an outside thread, preferably a self-cutting thread, at least at the apical end, then the inventive implant may advantageously be screwed into the bone without any prior measures.

In a particularly advantageous embodiment of the invention, the shaft area has at least one perforation arranged at the side, preferably connected by at least one channel to the bore. The shaft preferably has several perforations distributed uniformly over its circumference. In this embodiment, there is the possibility of administration of substances, cells or other material through the distal opening directly into different level areas of the osseous bed, i.e., any osseous tissue and, for example, the alveolar ridge, sinus, cyst lumen, osteomyelitic zone or fracture gap over the entire area or a portion of the implant or even beyond that. If the perforations are arranged in different compartments of the shaft area, targeted administration into the various levels of the osseous bed is advantageously possible, in particular when the various compartments of the shaft area can be sealed with respect to one another. For example, this may take place by means of a screw that can be screwed into the transitional area between the compartments or a stopper that can be attached there.

The shaft area preferably has at least one fastening means at the distal end in the bore. The fastening means may be, for example, an inside thread and/or a part of a plug or catch connection. For example, a line, an access tube, a screw, a tensioning spindle (mandrel) or a stopper may be attached to the fastening means. If a line, which may be a cannula or a hose, for example, or an access tube is attached to the fastening means, then reliable and targeted introduction or administration of substances, cells or other material through the distal opening into the bore is advantageously possible. By means of a screw mounted on the fastening means or a corresponding stopper, the outlet can be sealed and thus penetration of foreign bodies or microorganisms into the bore and optionally the surgical area between the individual treatments can be prevented. In special embodiments, a spacer sleeve or therapeutic sleeve, a prosthesis, a dental prosthesis and/or an adaptor may be attached to the fastening means for mounting the prosthesis or the dental prosthesis.

In an advantageous embodiment of the invention, the shaft area is also designed at least partially to be cylindrical and/or step-shaped.

The invention will now be explained in greater detail with reference to the figures as examples.

### BRIEF DESCRIPTION OF THE DRAWINGS

FIG. 1 shows a perspective view of one embodiment of the inventive implant with the therapeutic sleeve screwed onto it after implantation in a jawbone and

FIG. 2 shows a schematic diagram of a longitudinal section through another embodiment of the inventive implant.

### DESCRIPTION OF VARIOUS EMBODIMENTS OF THE INVENTION

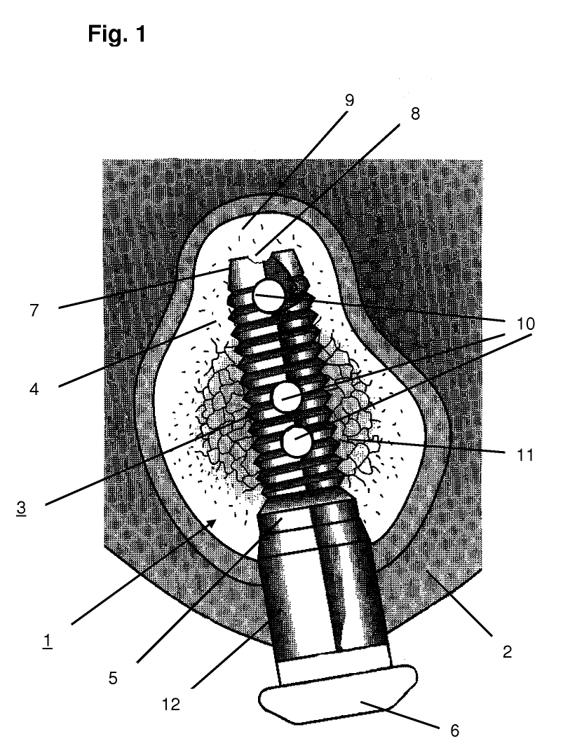

FIG. 1 shows as an example a perspective view of an inventive implant 1 after implantation in a jawbone 2. The implant 1 is designed to be essentially cylindrical and comprises primarily a shaft area 3 which has on the outside of its apical half a self-cutting outside thread 4 for screwing into the jawbone 2. On its distal end 5, a spacer sleeve or therapeutic sleeve 12 is screwed into an opening (not visible here) and is closed by means of a screw 6 (only the screw head being visible in this diagram). On its apical end 7 the shaft area 3 has an outlet 8 which is connected via a bore running along the longitudinal axis inside the shaft area 3 to the opening (closed here) on the distal end 5. When the screw 6 is removed, various substances, cells or other material can be introduced into the periapical space 9 through the opening in the therapeutic sleeve 12 via the outlet 8. For example, stem cells and/or growth factors may be introduced into the periapical space 9 to allow and/or support the formation of osseous tissue. In this way a sinus lift, for example, can be avoided.

The implant 1 also has perforations 10 in the shaft area 3 through which the therapeutic substances, cells or similar material can be introduced into the osseous tissue 11 surrounding the implant 1. Each perforation 10 is connected to the continuous bore via a channel, so that the material need be introduced only into the distal opening and/or the therapeutic sleeve 12 to pass through the perforations 10 into the osseous tissue 11. The perforations 10 are distributed uniformly over the circumference of the shaft area 3 of the implant 1, so that the surrounding osseous tissue 11 can also be treated uniformly with the substances and/or cells.

The inventive implant 1 thus allows administration of medications, stem cells or other material through the shaft area 3 directly during surgery and/or subsequently over a lengthy period of time in one or more sessions as inductive and/or adjuvant and/or therapeutic measure. Administration of the substances and/or cells specifically into the periapical area 9 is thus made possible, while the shaft area 3 retains its property of being an anchor in the osseous tissue 11. A post-implant therapeutic treatment of the surrounding tissue, especially at the apical end 7 of the implant by administration of medication, e.g., antibiotics or growth factors remains possible even after enossal integration of the implant 1. The implant 1 may be adapted to implantation in various bones, e.g., the jawbone, hip bone or upper thigh bone, depending on how it is used.

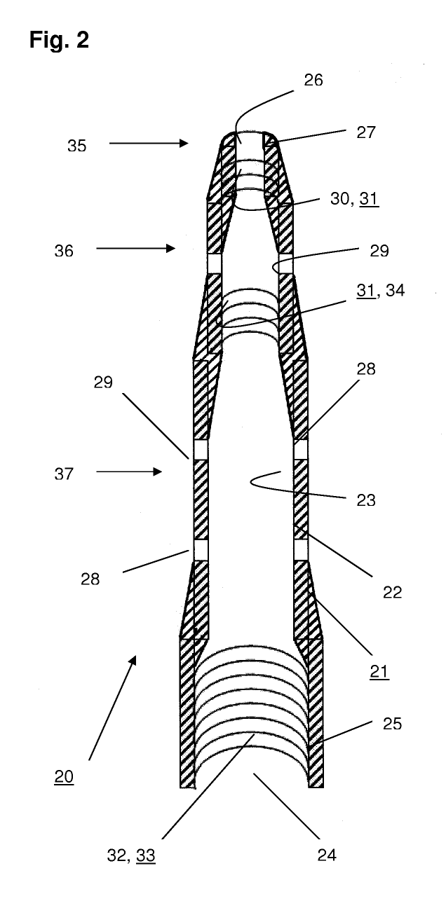

FIG. 2 shows as an example a schematic diagram of a longitudinal section through an inventive implant 20 that consists essentially of/comprises essentially a cylindrical shaft area 21. The shaft area 21 is formed by a hollow cylinder 22 designed with a step shape, having a continuous bore 23 in its interior. The bore 23 extends from the opening 24 on the distal end to an outlet 26 at the apical end 27 of the shaft area 21. The cylinder 22 has perforations 28 on its circumference, each perforation being connected by a channel 29 to the bore 23. The cylinder 22 may be completely or partially provided with an outside thread (not shown here) on its outer surface for screwing it into a bony structure.

At the apical end 27 of the shaft area 21 which forms the tip of the implant 20, an inside thread 30 is arranged in the bore 23 as a fastening element 31. For example, a line, an access tube, a screw, a tensioning spindle (mandrel) and/or a stopper may be attached to the fastening element 31. In the present exemplary embodiment, a screw or a screw mandrel can be screwed into the inside thread 30 so that the apical end 27 may either be closed or may be provided with, for example, an access line for introducing substances and/or cells into the periapical space. The interior cone of the distal end 25 of the shaft area 21 is also provided with an inside thread 32 as the fastening means 33 so that the opening 24 can be closed with a screw so that when the implant 20 heals and becomes fused with the osseous tissue, for example, no unwanted foreign material or microorganisms can enter the surgical area. While bone is growing into the implant, a spacer sleeve or a therapeutic sleeve may be applied to the fastening means 33 to facilitate the attachment of a prosthesis after the surrounding tissue has completely healed. If the implant 20 has healed in the bone and/or fused with it, then a spacer sleeve, a prosthetic device, a dental replacement or a corresponding adaptor may be screwed into the inside thread 32. The implant 20 then serves as an anchor for the prosthesis or the dental substitute in the bone. Then the surrounding osseous tissue remains accessible for administration of therapeutic substances even after implantation, via the opening 24 (after removal of the prosthesis, the dental substitute or the adaptor), the perforations 28 and especially the outlet 26. Furthermore, a tube or a cannula or the like may also be attached to the fastening means 33 to facilitate the introduction of material into the borehole 23.

The bore 23 has as an additional fastening element 31 another inside thread 34 near the apical end 27 so that a line, an access tube, a screw, a tensioning spindle (mandrel) and/or a stopper can be attached there. With the help of the fastening elements 31, the shaft area 21 and/or the bore 23 can be subdivided into different compartments 35, 36, 37. The individual compartments 35, 36, 37 can be sealed with respect to one another by the optional closing of the bore 23 by attaching a screw or stopper to one or more fastening elements 31, so that targeted administration to different levels of the surrounding tissue is possible through the respective perforations 28. For example, if the compartment 35 is closed by means of a screw that is screwed into the inside thread 30 and if a mandrel with an access line is screwed into the inside thread 34, then therapeutic substances and/or cells can be introduced in a targeted manner through the perforations of the compartment 36 into the corresponding level of the surrounding tissue. The same thing is of course also true of the compartment 37, whereby in this case the compartment 36 must be sealed by closing the bore 23 on the inside thread 34.

Other fastening elements and perforations may of course also be arranged in the shaft area 21. The inventive implant 20 can thus be used in a very flexible manner owing to the varied design options and may be adapted to different requirements and/or applications in the maxillary) surgical and orthopedic areas.

### LIST OF REFERENCE NUMERALS

1 implant
2 jawbone
3 shaft area
4 outside thread
5 distal end
6 screw
7 apical end
8 outlet
9 periapical space
10 perforations
11 osseous tissue
12 therapeutic sleeve
20 implant
21 shaft area
22 cylinder
23 bore
24 opening
25 distal end
26 outlet
27 apical end
28 perforations
29 channel
30 inside thread
31 fastening element
32 inside thread
33 fastening means/fastening structure
34 inside thread
35 Compartment
36 Compartment
37 Compartment

I claim:

1. An implant comprising
at least one shaft area for anchoring in a bony structure,
at least one opening at the distal end of the shaft area,
wherein the shaft area has a continuous bore extending from the opening to at least one outlet at the apical end.

2. The implant according to claim 1,
wherein the shaft area has at least one fastening element at the apical end in the bore.

3. The implant according to claim 2,
wherein a line, an access tube, a screw, a tension mandrel or a stopper can be attached to the fastening element.

4. The implant according to claim 2,
wherein the fastening element is an inside thread and/or a part of a plug or catch connection.

5. The implant according to claim 3,
wherein the fastening element is an inside thread and/or a part of a plug or catch connection.

6. The implant according to claim 3,
wherein the line is a cannula or a tube.

7. The implant according to claim 1,
wherein the shaft area has, at least at the apical end, an outside thread.

8. The implant according to claim 7, wherein the outside thread is self-cutting.

9. The implant according to claim 1, wherein the shaft area has at least one lateral perforation.

10. The implant according to claim 9, wherein said perforation is connected to the bore by at least one channel.

11. The implant according to claim 9,
wherein perforations are provided in various compartments of the shaft area.

12. The implant according to claim 11,
wherein the various compartments of the shaft area can be sealed with respect to one another.

13. The implant according to claim 1, wherein the shaft area has at least one fastening structure at the distal end in the bore.

14. The implant according to claim 13, wherein a line, an access tube, a screw, a tension mandrel or a stopper can be attached to the fastening structure.

15. The implant according to claim 14, wherein the line is a cannula or a hose.

16. The implant according to claim 13, wherein
a spacer sleeve or a therapeutic sleeve, a prosthesis,
a dental substitute and/or an adaptor for applying the prosthesis or the dental substitute can be attached to the fastening structure.

17. The implant according to claim 9, wherein the shaft area has at least one fastening structure at the distal end in the bore.

18. The implant according to claim 17, wherein a line, an access tube, a screw, a tension mandrel or a stopper can be attached to the fastening structure.

19. The implant according to claim 18, wherein the line is a cannula or a hose.

20. The implant according to claim 1, wherein the shaft area is designed to be at least partially cylindrical and/or step-shaped.
